(19) United States
(12) Patent Application Publication
(10) Pub. No.: US 2018/0355390 A1
(43) Pub. Date: Dec. 13, 2018

(54) Method to produce L-methionine by a fermentative production

(71) Applicant: Evonik Degussa GmbH, Essen (DE)

(72) Inventors: Laetitia FAURE, Les Martres De Veyre (FR); Cédric Colomb, Ennezat (FR); Antoine Sevenier, Pont du Chateau (FR); Michel Chateau, Riom (FR)

(73) Assignee: Evonik Degussa GmbH, Essen (DE)

(21) Appl. No.: 15/774,267

(22) PCT Filed: Jan. 8, 2016
(86) PCT No.: PCT/IB2016/000122

§ 371 Date: May 7, 2018

(51) Int. Cl. C12P 13/12; C07C 319/28

(52) U.S. Cl. CPC C12P 13/12; C07C 319/28

(57) ABSTRACT

A method can produce L-methionine and/or a derivative of L-methionine. The method includes: culturing a microorganism for the fermentative production of L-methionine and/or a L-methionine derivative in a culture medium comprising a carbon source, a sulphur source and nitrogen source to produce L-methionine and/or a L-methionine derivative; recovering the L-methionine or L-methionine derivative obtained from the culturing; adding an alcohol solvent or acetonitrile to the L-methionine and/or the L-methionine derivative thereof containing fraction obtained from the recovering to precipitate the L-methionine and/or L-methionine derivative; and recovering L-methionine and/or L-methionine derivative.

### FIELD OF THE INVENTION

The present invention relates to a method for the fermentative production of L-methionine and/or its derivatives with a high degree of purity.

### PRIOR ART

Sulphur-containing compounds such as methionine, cysteine, homocysteine or 5-methyladenosylmethionine are critical to cellular metabolism and are produced industrially to be used as food or feed additives and pharmaceuticals. In particular methionine, an essential amino acid, which cannot be synthesized by animals, plays an important role in many body fonctions. Aside from its role in protein biosynthesis, methionine is involved in transmethylation and in the bioavailability of selenium and zinc. Methionine is also directly used as a treatment for disorders like allergy and rheumatic fever. Nevertheless, most of the methionine that is produced is added to animal feed.

With the decreased use of animal-derived proteins as a result of BSE and chicken flu, the demand for pure methionine has increased. Commonly, D,L-methionine is produced chemically from acrolein, methyl mercaptan and hydrogen cyanide. However, the racemic mixture does not perform as well as pure L-methionine (Saunderson, C. L., 1985). Additionally, although pure L-methionine can be produced from racemic methionine, for example, through the acylase treatment of N-acetyl-D,L-methionine, this dramatically increases production costs. Accordingly, the increasing demand for pure L-methionine coupled with environmental concerns render microbial production of methionine an attractive project.

However, the implementation of these methods requires the availability of suitable microorganisms for producing methionine by fermentation on a carbon sole. Industrially effective solutions have been published, particularly in patent applications WO 2005/111202, WO 2007/017710, WO 2007/077041 and WO 2009/043803. Other microorganisms producing methionine are also described in the applications WO 2004/038013, WO 2006/001616, WO 2006/138689 and WO 2007/012078.

Different methods are proposed to recover the L-methionine from fermentation broth. Because of the low solubility of methionine under normal fermentation conditions, its separation from whole cells and other fermentation broth components is a major issue which needs to be solved to be able to produce L-methionine economically.

WO 2005/007862 discloses a method when solubility of methionine in fermentation broth or in fermentation supernatant is increased by adjusting the pH to an acidic or basic pH and/or by increasing the temperature. Then, the methionine can be selectively crystallized. Methionine solubility is manipulated to make a purified methionine end product which can be dried and granulated for use in the animal feed sector.

WO 2011/045377 and WO2013/083934 relate a novel liquid or crystalline methionine composition obtained by fermentation. The methionine and other component content allow an easier purification, particularly for obtaining a solid product having a higher content of methionine compared to the dry residue of liquid composition.

US2012/0178966 discloses the recovery of methionine in the same time than carbon and nitrogen contained in the supernatant. Methionine is produced by a two-step process comprising first the synthesis of a precursor of L-methionine, either O-acetylhomoserine or O-succinylhomoserine, followed by a second step which is the enzymatically transformation into L-methionine.

Because these methods includes many steps, and because these methods does not provide a high grade purity L-methionine, the applicant has found an effective method to produce high grade L-methionine.

### DETAILED DESCRIPTION OF THE INVENTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting, which will be limited only by the appended claims.

All publications, patents and patent applications mentioned herein are cited for the purpose of describing and disclosing the protocols, reagents and vectors that are reported in the publications and that might be used in connection with the invention.

Furthermore, the practice of the present invention employs, unless otherwise indicated, conventional microbiological and molecular biological techniques within the skill of the art. Such techniques are well known to the skilled worker, and are explained fully in the literature.

In the claims that follow and in the consecutive description of the invention, except where the context requires otherwise due to express language or necessary implication, the word “comprise”, “contain”, “involve” or “include” or variations such as “comprises”, “comprising”, “containing”, “involved”, “includes”, “including” are used in an inclusive sense, i.e. to specify the presence of the stated features but not to preclude the presence or addition of further features in various embodiments of the invention.

The term “methionine” and “L-methionine” designate the essential sulphur-containing amino-acid with chemical formula HO₂CCH(NH₂)CH₂CH₂SCH₃ and CAS number 59-51-8 or 63-68-3 for the specific L-isomer.

“Derivatives of methionine” refers to molecules analogs to methionine which present the same chemical backbone but differ from methionine with at least one chemical group. In this invention, preferred methionine derivatives are N-acetyl methionine (NAM), S-adenosyl methionine (SAM) and hydroxy-methionine.

The term “microorganism”, as used herein, refers to a bacterium, yeast or fungus which is not modified artificially. Preferentially, the microorganism is selected among Enterobacteriaceae, Bacillaceae, Streptomycetaceae and Corynebacteriaceae. More preferentially the microorganism is a species of Escherichia, Klebsiella, Pantoea, Salmonella, or Corynebacterium. Even more preferentially the microorganism used according to the invention is either the species Escherichia coli or Corynebacterium glutamicum.

The terms “improved methionine production”, “improve methionine production” and grammatical equivalents thereof, as used herein, refer to an increased methionine/carbon source yield (ratio of gram/mol methionine produced per gram/mol carbon source consumed that it can be expressed in percent) and/or an improved purity of produced methionine. In this invention, the purity of the produced methionine may be increased by precipitating L-methionine by a solvent. Methods for determining the amount of carbon source consumed and of methionine produced are well known to those in the art. The yield of produced methionine are higher in the method of the invention.

The terms “microorganism optimised for the fermentative production of methionine” or “recombinant microorganism” as employed in this disclosure refer to microorganisms evolved and/or genetically modified to present an improved methionine production in comparison with the endogenous production of the corresponding wild-type microorganisms. Such microorganisms “optimised” for methionine production are well known in the art, and have been disclosed in particular in patent applications WO2005/111202, WO2007/077041, WO2009/043803, WO2012/098042, WO2013/001055, WO2013/190343, WO2015/028674 and WO2015/028675.

According to the invention the terms “fermentative production”, “culture” or “fermentation” are used interchangeably to denote the growth of microorganism. This growth is generally conducted in fermenters with an appropriate culture medium adapted to the microorganism being used and containing at least one simple carbon source, and if necessary co-substrates like a source of sulphur and a source of nitrogen.

An “appropriate culture medium” designates a medium (e.g., a sterile, liquid media) comprising nutrients essential or beneficial to the maintenance and/or growth of the cell such as carbon sources or carbon substrates, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids and vitamins.

The term “carbon source” or “carbon substrate” or “source of carbon” according to the present invention denotes any source of carbon that can be used by those skilled in the art to support the normal growth of a microorganism, including monosaccharides (such as glucose, galactose, xylose, fructose or lactose), oligosaccharides, disaccharides (such as sucrose, cellobiose or maltose), molasses, starch or its derivatives, hemicelluloses and combinations thereof. An especially preferred simple carbon source is glucose. Another preferred simple carbon source is sucrose. The carbon source can be derived from renewable feed-stock. Renewable feed-stock is defined as raw material required for certain industrial processes that can be regenerated within a brief delay and in sufficient amount to permit its transformation into the desired product. Vegetal biomass treated or not, is an interesting renewable carbon source.

The term “source of sulphur” according to the invention refers to sulphate, thiosulfate, hydrogen sulphide, dithionate, dithionite, sulphite, methylmercaptan, dimethylsulfide and other methyl capped sulphides or a combination of the different sources. More preferentially, the sulphur source in the culture medium is sulphate or thiosulfate or a mixture thereof.

The terms “source of nitrogen” corresponds to either an ammonium salt or ammoniac gas. The nitrogen source is supplied in the form of ammonium or ammoniac.

### Culture Conditions

The invention is related to a method of production of L-methionine and/or its derivatives comprising the followings steps:

i) culturing a microorganism optimized for the fermentative production of methionine in an appropriate culture medium comprising a carbon source, a sulphur source and nitrogen source to produce L-methionine;
ii) recovering the L-methionine obtained at the step i);
iii) adding an alcohol solvent or acetonitrile to the methionine containing fraction obtained at step ii) to precipitate the L-methionine; and
iv) recovering L-methionine.

The step i) according to the invention comprises culturing a microorganism.

In particular the microorganism are fermented at a temperature between 20° C. and 55° C., preferentially between 25° C. and 40° C., and more specifically about 30° C. for C. glutamicum and about 37° C. for E. coli.

For E. coli, the culture medium can be of identical or similar composition to an M9 medium (Anderson, 1946), an M63 medium (Miller, 1992); or a medium such as defined by Schaefer et al., (1999).

For C. glutamicum, the culture medium can be of identical or similar composition to BMCG medium (Liebl et al., 1989) or to a medium such as described by Riedel et al., (2001).

Preferably the microorganism at the step i) of the method according to the invention is the sole microorganism. That mean only one strain of microorganism is used, the strain producing L-methionine. No microorganism producing L-methionine precursor is used according to the method of the invention.

Preferably, the method according to the invention does not comprised any enzymatically processing of an L-methionine precursor, at any step.

The step ii) according to the invention comprises recovering L-methionine.

The action of recovering L-methionine at the step ii) should be understood by recovering L-methionine and/or its derivatives from the culture medium. It designates the action of recovering L-methionine and/or one of its derivatives, in particular N-acetyl methionine (NAM) and 5-adenosyl methionine (SAM) and all other derivatives that may be useful. The methods for the recovery and purification of the produced compounds are well known to those skilled in the art (see in particular WO2005/007862, WO2005/059155). Preferably, the step ii) of recovering L-methionine and/or its derivatives comprises a step of concentration of L-methionine and/or its derivatives in the fermentation broth.

The step ii) according to the invention may comprise a plurality of steps like for example a clarification step of the fermentation medium, followed or not by a concentration step of L-methionine.

A clarification step of the fermentation medium means clarifying the fermentation medium and removing the insoluble and soluble organic impurities from said fermentation medium in order to obtain a liquid crude methionine containing fraction. The clarification of the medium is carried out by any method known as such by those skilled in the art, which method is chosen, for example, from the group consisting of flocculation, decanting, membrane techniques (microfiltration, ultrafiltration, diafiltration, nano-filtration and reverse osmosis) and centrifugation. The removal of the soluble organic impurities is carried out by any method known as such by those skilled in the art, which method is chosen, for example, from the group consisting of ultrafiltration, heat treatment, treatment with an adsorbent of activated carbon type, and enzymatic hydrolysis. The removal of these soluble impurities makes it possible to ensure that the cooked mass behaves correctly during precipitation. Indeed, without this step, the cooked mass has a pasty homogeneous appearance, producing very fine crystals and penalizing the separation and washing of said crystals. Consequently, the purity of the crystals is reduced.

The removal of soluble impurities is then followed by the crystallization step with or without a preliminary pre-concentration step.

Crystallization step consists in crystallizing the methionine so as to recover the crude methionine in solid form. This crystallization step can be carried out by means of technology chosen from the group consisting of crystallization by cooling, crystallization by evaporation-crystallization and adiabatic crystallization. The applicant company recommends using evaporation-crystallization. If evaporation-crystallization is selected, the applicant company recommends pre-concentrating the crude methionine solution by vacuum evaporation using a falling-film evaporator in order to approach supersaturation. The pre-concentrated solution is therefore transferred into a crystallizer of Draft tube type, for example, so as to be further concentrated and crystallized therein.

The methionine solubility at 35° C. is approximately 70 g/L. If the solution is concentrated to approximately 250 g/L, under a vacuum ensuring a temperature of 35° C., the solid methionine recovery yield is >70%. Under these conditions, and by virtue of the removal of the soluble impurities, the solid methionine crystallizes in the form of spongy spheres which are easy to separate from the mother liquors.

The step iii) according to the invention comprises adding an alcohol solvent or acetonitrile to the methionine containing fraction obtained at step ii) to precipitate the L-methionine. This step is a precipitation step of the L-methionine or its derivatives. The alcohol solvent used at step iii) may be a chosen among ethanol, isopropanol or a mixture thereof. Preferably, the alcohol solvent used at step iii) is ethanol.

Preferably, at the step iii) the ratio of the solvent/(methionine containing fraction+solvent) is comprised from 50 to 80% by weight, preferably from 65 to 75%. It is noted that the term solvent used here means alcohol solvent or acetonitrile.

Preferably the residual solubility of L-methionine in the supernatant after step iii) is from 5 to 6 g·L⁻¹ at room temperature, more preferably from 5.2 to 5.9 g·L⁻¹.

In some exemplary embodiment of the invention, the working temperature of the step iii) is comprised from 2° C. to 50° C., preferably from 10° C. to 40° C., even more preferably from 18° C. to 40° C.

Recovery of precipitated methionine may be achieved by any means known by the man skilled in the art such as decantation or centrifugation, preferably by decantation.

The step iv) according to the invention is the recovering L-methionine. The step iv) could be different from the previous step ii) and can use different methods from those used at step ii).

This step iv) may comprise the separation of the resulting solid crude methionine, in washing it and in drying it. The solid crude methionine is recovered by any method known as such by those skilled in the art, which method is chosen, for example, from the group consisting of centrifugation, suction filtration and frontal filtration (on a drum, on a filter press, etc.). In the latter case, the crude methionine is therefore retained on a 40 μm cloth and the mother liquors pass through. The crude methionine cake is then washed with 1 to 10 BV of water, preferably demineralized water. The solid crude methionine is then dried and optionally milled in order to classify the particles.

The amount of product in the fermentation medium or in the different downstream process fluxes such as supernatant, permeate or mother liquors of crystallization can be determined using a number of methods known in the art, for example, high performance liquid chromatography (HPLC) or gas chromatography (GC). For example the quantity of methionine obtained in the medium is measured by HPLC after OPA/FMOC derivatization using L-methionine (Fluka, Ref 64319) as a standard.

Preferably the concentration of L-methionine after the step iv) is at least 180 g·L⁻¹, preferably at least 200 g·L⁻¹, and particularly comprised from 180 to 300 g·L⁻¹, more preferably from 220 to 300 g·L⁻¹.

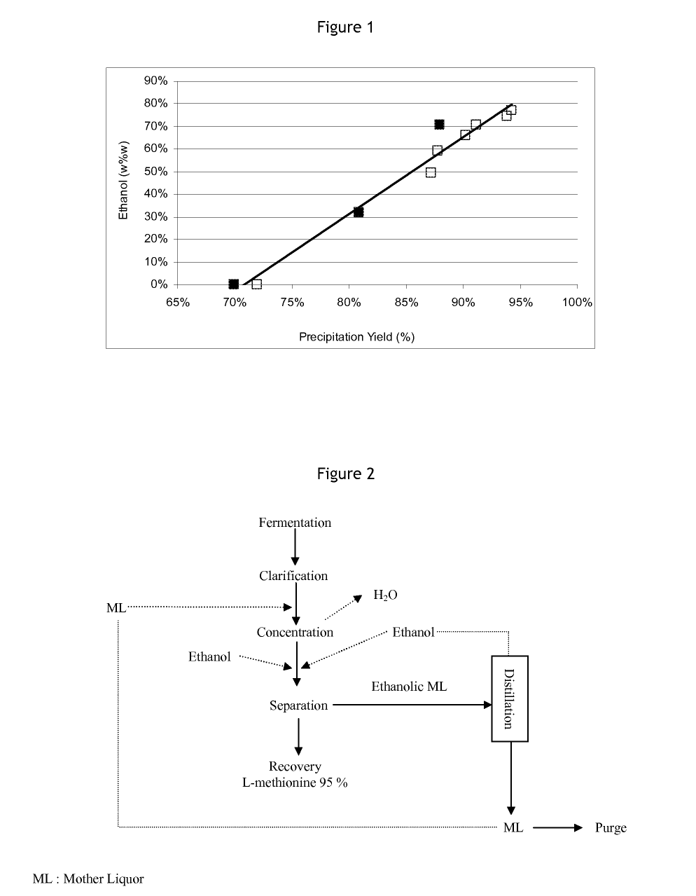

The FIG. 1 represents the precipitation yield in function of volume of ethanol used (empty squares: theoretical yield and solid squares: experimental results).

The FIG. 2 represents an exemplary process of methionine precipitation with recycling mother liquor.

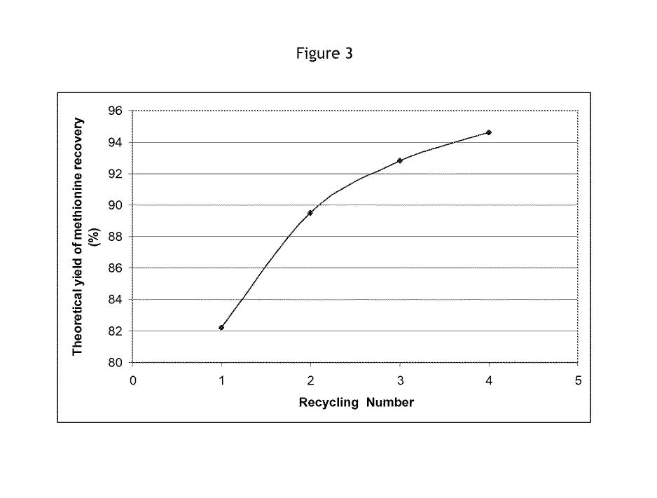

The FIG. 3 represents the theoretical yield of methionine recovery in function of number of ethanolic mother liquor recycling.

### EXAMPLES

The present invention is further defined in the following examples. It should be understood that these examples, while indicating preferred embodiments of the invention, are given by way of illustration only. From above disclosure and these examples, the man skilled in the art can make various changes of the invention to adapt it to various uses and conditions without modify the essentials means of the invention.

In particular, examples show modified Escherichia coli (E. coli) strains, but these modifications can easily be performed in other microorganisms of the same family.

Escherichia coli belongs to the Enterobacteriaceae family, which comprises members that are Gram-negative, rod-shaped, non-spore forming and are typically 1-5 μm in length. Most members have flagella used to move about, but a few genera are non-motile. Many members of this family are a normal part of the gut flora found in the intestines of humans and other animals, while others are found in water or soil, or are parasites on a variety of different animals and plants. E. coli is one of the most important model organisms, but other important members of the Enterobacteriaceae family include Klebsiella, in particular Klebsiella terrigena, Klebsiella planticola or Klebsiella oxytoca, and Salmonella.

Moreover, several patent applications point out that optimization for methionine production can easily be applied in E. coli and in Corynebacterium glutamicum without undue experimentation.

### Example 1: Methionine Production by Fermentation Process

Step i): Culture of the Microorganism

Culturing and fermentation of the L-methionine producing bacteria can be performed according to any method known in the art, and in particular to the method described in the the example 4 of patent application WO2013/190343.

Notably, culture conditions and media compositions are identical except for the time of fermentation which here is continued at 37° C. until obtaining a methionine concentration of 65 g·L⁻¹ (the culture time is between 30 to 35 hours).

Step ii) Recovering the L-Methionine
The removal of the insoluble Organic Impurities (biomass) is carried out in a first step by tangential filtration on a membrane having a pore diameter of 100 nm, at between and 80° C. (membrane of ceramic type with a channel diameter of 3.5 mm). The temperature is preferentially maintained at 40° C. with a transmembrane pressure of 1 bar and diafiltration with 20% of demineralized water. Under these conditions, the average flow rate is 30 L/h/m² and the permeate obtained is clear and bright. The permeate, freed of the biomass and of the insoluble particles, still contains soluble organic impurities, in particular soluble sugars and proteins that it is advisable to remove before the next step.
The objective of this second step is to remove soluble organic impurities (soluble sugars like polysaccharides and macromolecules) contained in the fermentation must. This is because these impurities negatively affect the behaviour of the cooked mass (homogeneous, bound appearance, very fine crystals) and the recovery of the solid phase during the L-methionine crystallization step.

This removal can be carried out by ultrafiltration on a ceramic membrane which has a cut-off threshold of 5 kDa. At 40° C., the filtration flow rate is on average 25 L/h/m², and approximately 70% of the macromolecules are retained in the retentate.

The demineralized solution is pre-concentrated by evaporation of the water at 50° C. on a Wiegand® falling-film vacuum evaporator. The concentration factor is about from 2 to 5 depending on the initial concentration of L-methionine. It is in this case equal to 3 so as to approach super-saturation at 50° C. (80 g/L). The pre-concentrated solution is then transferred into a forced-circulation evapocrystallizer and is further concentrated and crystallized therein under vacuum (50 mbar) at approximately 35° C. The concentration factor applied in this evapocrystallizer is approximately 3, so as to achieve 240 g/L.

Step iii) Precipitating with Solvent

Different solvents were assayed on the fermentation concentrated supernatant issued from step ii) with the same ratio of 70% (solvent/(methionine containing fraction+solvent), in weight). The solvents permitting to attain the lower residual solubility of methionine are ethanol and isopropanol (Table 1).

TABLE 1
Residual solubility of L-methionine in function
of used solvent and working temperature.

Residual solubility
L-Methionine (g · L⁻¹)

| | Solvent/Working | Room | |
|---|---|---|---|
| | temperature | Temperature | 4° C. |
| Reference | None | 65 | nd* |
| Comparative | Methanol | 7-8 | 5-6 |
| example | | | |
| invention | Ethanol | 5-6 | 4-5 |
| invention | Isopropanol | 5-6 | 4-5 |
| invention | Acetonitrile | 5.5-6.5 | nd* |
| Comparative | 2-Butanone | >15 | nd* |
| example | | | |

*not determined

Different percentages of the ethanol as solvent were assayed on the fermentation supernatant issued from step ii). The theoretical yield of recovery of L-methionine with different percentages of ethanol as solvent have put in line with the results. The FIG. 1 shows the experimental and theoretical results.

The fermentation supernatant issued from step ii) was concentrated to approximately 250 g·L⁻¹ and ethanol has been added at different proportions 0%, 30% and 70% (corresponding to the ratio solvent/(methionine containing fraction+solvent) in weight). The theoretical yield of recovery has been determined in function of ethanol content. The FIG. 1 demonstrates that the experimental data are in accordance with theoretical values (The solubility of L-meth at 37° C. is approximately 70 g·L⁻¹).

Step iv) Recovering the L-Methionine

The formed crystals after step iii) are recovered by separation on a Choquenet® filter press and washing with one volume of demineralized water per volume of cake. The crystals are dried on a fluidized bed at 45° C. (of the Aeromatic® type). Under these conditions, the L-methionine recovery yield is >80% for a purity >85%/dry.

### Example 2: Increased Recovery of L-Methionine with One Step of Precipitation Realised with Ethanol

Following the protocol of example 1, a fermentation broth derived from the implementation described above and containing L-methionine has been purified. The biomass formed during the fermentation has been separated off completely. The insoluble material was removed from the fermentation broth by centrifugation. The methionine concentration at 37° C. in the supernatant was closed to 65 g·L⁻¹. The solution is concentrated to approximately 200 g·L⁻¹ after the step ii), under vacuum (300 mbars) ensuring a temperature of 80° C. (by using rotavapor).

After cooling to room temperature, ethanol (70% W/W) was added to complete the precipitation of the methionine. The crystals are recovered by filtration. The filter cake is washed with ethanol at 70% (100 mL) and then dried by lyophilization.

One precipitation step with ethanol permits to increase the crystallization yield from 67.5 to 87% (Table 2).

TABLE 2
Precipitation yield without or with
one ethanol (70%) precipitation step.

| | Without Ethanol | With one step of Ethanol |
|---|---|---|
| | Precipitation | precipitation |
| Crystallization yield | 67.5% | 87% |

### Example 3: Increased Recovery of L-Methionine with Several Steps of Precipitation Realised with Ethanol

The ethanol can be used as described in the example 2 and the primary mother liquor obtained from the distillation step can be used in loop (FIG. 2). Ethanol is recycled for the precipitation step. One part of mother liquor is recycling to increase the yield. The methionine yield can be increased to 95% if 60% of mother liquor is recycled (FIG. 3).

### REFERENCES

Saunderson, 1985 British Journal of Nutrition 54: 621-633.
Anderson, 1946, Proc. Natl. Acad. Sci. USA 32:120-128.
Miller, 1992, Laboratory Manual and Handbook for Escherichia coli and Related Bacteria, Cold Spring Harbor Laboratory Press, Cold Spring Harbor, N.Y.
Schaefer et al. 1999, Anal. Biochem. 270: 88-96.
Liebl et al., 1989, Appl. Microbiol. Biotechnol. 32: 205-210.
Riedel et al., 2001, J. Mol. Microbiol. Biotechnol. 3: 573-583.

1: A method for the a fermentative production of L-methionine and/or a derivative thereof, the method comprising:
i) culturing a microorganism for the fermentative production of L-methionine and/or a derivative thereof in a culture medium comprising a carbon source, a sulphur source and nitrogen source to produce L-methionine and/or a derivative thereof;
ii) recovering the L-methionine and/or a derivative thereof obtained from said i) culturing;
iii) adding an alcohol solvent or acetonitrile to the L-methionine and/or a derivative thereof containing fraction obtained from said ii) recovering to precipitate the L-methionine and/or a derivative thereof; and
iv) recovering L-methionine and/or a derivative thereof.

2: The method according to claim 1, wherein the microorganism of said i) culturing is the sole microorganism.

3: The method according to claim 1, which does not comprise
any enzymatic processing of an L-methionine precursor.

4: The method according to claim 1, wherein
said ii) recovering comprises a clarification of the fermentation medium, optionally followed by a crystallization of L-methionine and/or a derivative thereof.

5: The method according to claim 1,
wherein the alcohol solvent of said iii) adding is at least one member selected from the group consisting of ethanol and isopropanol.

6: The method according to claim 1, wherein
the alcohol solvent of said iii) adding is ethanol.

7: The method according to claim 1, wherein,
in said iii) adding, ratio solvent/(methionine containing fraction+solvent) is from 50 to 80% by weight.

8: The method according to claim 1, wherein
in said iii) adding, a residual solubility of L-methionine in a supernatant after said iii) adding is from 5 to 6 g·L⁻¹ at room temperature.

9: The method according to claim 1, wherein,
in said iii) adding, ratio solvent/(methionine containing fraction+solvent) is from 65 to 78% by weight.
